(19) United States
(12) Patent Application Publication
(10) Pub. No.: US 2024/0011044 A1
(43) Pub. Date: Jan. 11, 2024

(54) PLANT REGULATORY ELEMENTS AND USES THEREOF

(71) Applicant: University of Freiburg, Freiburg (DE)

(72) Inventors: Thomas Ott, Merzhausen (DE); Chao Su, Bad Krozingen (DE)

(21) Appl. No.: 18/346,809

(22) Filed: Jul. 4, 2023

Related U.S. Application Data
(60) Provisional application No. 63/367,703, filed on Jul. 5, 2022.

(51) Int. Cl. C12N 15/82; C12N 9/18; C12N 15/52

(52) U.S. Cl. CPC C12N 15/8227; C12N 9/18; C12Y 301/01011; C12N 15/52; C12N 15/8237

(57) ABSTRACT

The present invention provides novel DNA molecules and constructs, including their nucleotide sequences, useful for modulating gene expression in plants and plant cells. The invention also provides transgenic plants, plant cells, plant parts, seeds, and commodity products comprising the DNA molecules operably linked to heterologous transcribable polynucleotides, along with methods of their use.

### CROSS REFERENCE TO RELATED APPLICATION

This application claims the benefit of U.S. Provisional Application No. 63/367,703 filed Jul. 5, 2022, which is hereby incorporated herein by reference in its entirety.

### INCORPORATION OF SEQUENCE LISTING

The sequence listing that is contained in the file named “AGOE010US ST26.xml”, which is 12.5 KB (as measured in Microsoft Windows®) and was created on Jun. 7, 2023, is filed herewith by electronic submission and is incorporated by reference herein.

### FIELD OF THE INVENTION

The invention relates to the field of plant molecular biology and plant genetic engineering, and DNA molecules useful for modulating gene expression in plants.

### BACKGROUND

Regulatory elements are genetic elements that regulate gene activity by modulating the transcription of an operably linked transcribable polynucleotide molecule. Such elements include promoters, leaders, introns, and 3′ untranslated regions and are useful in the field of plant molecular biology and plant genetic engineering.

### SUMMARY OF THE INVENTION

The present invention provides novel gene regulatory elements for use in plants. The present invention also provides DNA constructs comprising the regulatory elements. The present invention also provides transgenic plant cells, plants, and seeds comprising the regulatory elements. The sequences may be provided operably linked to a transcribable polynucleotide molecule. In one embodiment, the transcribable polynucleotide molecule may be heterologous with respect to a regulatory sequence provided herein. A regulatory element sequence provided by the invention thus may, in particular embodiments, be defined as operably linked to a heterologous transcribable polynucleotide molecule. The present invention also provides methods of making and using the regulatory elements, the DNA constructs comprising the regulatory elements, and the transgenic plant cells, plants, and seeds comprising the regulatory elements operably linked to a transcribable polynucleotide molecule.

Thus, in one aspect, the present invention provides a DNA molecule comprising a DNA sequence selected from the group consisting of: a) a sequence having at least about 85 percent sequence identity to any of SEQ ID NO: 1; b) a sequence comprising SEQ ID NO: 1; and c) a fragment of SEQ ID NO: 1 or a fragment having at least 85 percent sequence identity to the fragment of SEQ ID NO: 1, wherein the fragment has gene-regulatory activity; wherein the sequence is operably linked to a heterologous transcribable polynucleotide molecule. In specific embodiments, the DNA molecule comprises at least about 90 percent, at least about 95 percent, at least about 98 percent, or at least about 99 percent sequence identity to the DNA sequence of SEQ ID NO: 1. In certain embodiments, the DNA molecule has at least 87 percent sequence identity to a fragment of SEQ ID NO: 1 if the fragment is less than 115 nucleotides. In certain embodiments of the DNA molecule, the DNA sequence comprises a regulatory element. In some embodiments, the regulatory element comprises a promoter. In further embodiments, the DNA molecule has gene regulatory activity, such as promoter activity or symbiotic-specific pectin methyl esterase (SyPME) promoter activity. In certain embodiments, the heterologous transcribable polynucleotide molecule comprises a gene of agronomic interest. In some embodiments, the gene of agronomic interest is a gene encoding pectin methylesterase having pectin de-methylesterification activity. In particular embodiments, the DNA sequence provides expression of the heterologous transcribable polynucleotide molecule in response to an external stimulus. In some embodiments, the DNA sequence provides expression of said heterologous transcribable polynucleotide molecule in a root hair cell, within the cortex of nodule primordia, a mature nodule, within a nodular infection zone in young, mature, or indeterminate nodules.

The invention also provides a transgenic plant cell comprising a heterologous DNA construct provided by the invention, including a sequence of SEQ ID NO: 1, or a fragment or variant thereof, wherein said sequence is operably linked to a heterologous transcribable polynucleotide molecule. In certain embodiments, the transgenic plant cell is a monocotyledonous plant cell. In other embodiments, the transgenic plant cell is a dicotyledonous plant cell.

Further provided by the invention is a transgenic plant, or part thereof, comprising a DNA molecule as provided herein, including a DNA sequence selected from the group consisting of: a) a sequence having at least 85 percent sequence identity to SEQ ID NO: 1; b) a sequence comprising SEQ ID NO: 1; and c) a fragment of SEQ ID NO: 1 or a fragment having at least 85 percent sequence identity to the fragment of SEQ ID NO: 1, wherein the fragment has gene-regulatory activity; wherein the sequence is operably linked to a heterologous transcribable polynucleotide molecule. In specific embodiments, the transgenic plant may be a progeny plant of any generation that comprises the DNA molecule, relative to a starting transgenic plant comprising the DNA molecule. Still further provided is a transgenic seed comprising a DNA molecule according to the invention.

In yet another aspect, the invention provides a method of producing a commodity product comprising obtaining a transgenic plant or part thereof according to the invention and producing the commodity product therefrom. In one embodiment, a commodity product of the invention is protein concentrate, protein isolate, grain, starch, seeds, meal, flour, biomass, or seed oil. In another aspect, the invention provides a commodity produced using the above method. For instance, in one embodiment the invention provides a commodity product comprising a DNA molecule as provided herein, including a DNA sequence selected from the group consisting of: a) a sequence with at least 85 percent sequence identity to SEQ ID NO: 1; b) a sequence comprising SEQ ID NO: 1; and c) a fragment of SEQ ID NO: 1, wherein the fragment has gene-regulatory activity; wherein the sequence is operably linked to a heterologous transcribable polynucleotide molecule.

In still yet another aspect, the invention provides a method of expressing a transcribable polynucleotide molecule that comprises obtaining a transgenic plant or part thereof according to the invention, such as a plant comprising a DNA molecule as described herein, and cultivating the plant, wherein the transcribable polynucleotide in the DNA molecule is expressed.

Throughout this specification and the claims, unless the context requires otherwise, the word “comprise” and its variations, such as “comprises” and “comprising,” will be understood to imply the inclusion of a stated composition, step, and/or value, or group thereof, but not the exclusion of any other composition, step, and/or value, or group thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows pectins differentially modified in infected cells. 14 days old M. truncatula nodules were embedded in LR Gold and hybridized with the respective antibodies (red). DNA was counterstained with DAPI (blue). (Panel A) LM20 labelled all the cell wall structure. (Panel B) LM19 showed specific staining of transcellular nodular infection threads (ITs; white arrowheads), additionally LM19 also labelled epidermal cells and outer cortex cells. (Panels C-D) Immunofluorescence labeling (Red) with the antibodies LM19 (Panel C) and LM20 (Panel D) with roots (5 days post inoculation) sections embedded in LR white. Arrows indicate infection thread. (Panels E-F″) CLEM analysis with LM19 (Panels E-E″) and LM20 (Panels F-F″). Scale bars indicate 50 1.tm in A-D; 2.5 μm in Panels E-F″. TEM: Transmission electron microscopy.

FIG. 2 shows un-esterified pectins are concentrated at the ITs penetration sites. (Panel A) Scanning electron microscopy (SEM) and (Panel B) transmission electron microscopy (TEM) images of infected cells inside nodule. Scale bars indicate 10 μm. (Panel C) Double immune-gold labeling with LM19 (12 nm) and LM20 (5 nm) at the infection thread passage site inside nodule. Scale bars indicate 0.5 μm. (Panel D) Immunofluorescence labeling (red) with the antibodies 2F4, DNA was counterstained with DAPI (blue). Scale bars indicate 10 μm. Arrow heads indicate infection thread (IT, in Panel A); arrows indicate IT penetration sites (in Panels A-D).

FIG. 3 shows localization analysis of SyPME and NPL inside the nodule. (Panels A-C) The localization of SyPME-GFP in a transformed nodule section. SyPME-GFP signal is mainly located in IT and strongly accumulates at the penetration sites (Panels A-C, indicated by arrowheads) and the tip of IT (Panels D-F, indicated by stars). SyPME-GFP is also slightly concentrated in the local region near IT (Panels D-F, indicated by arrows). The scale bars indicate 5 μm. (Panels G-L) The localization of NPL-GFP in a transformed nodule section. NPL-GFP signal strongly accumulates at the tip of IT (Panels G-I, indicated by stars) and slightly accumulates at the local region near the tip of IT (Panels G-I, indicated by arrows, the inserted plot profile in E and H indicate the GFP signal intensity marked by the red line (near to arrow) within the image). NPL-GFP does not concentrate at the IT penetrations sits (Panels J-L, indicated by arrowheads). Scale bars indicate 5 μm.

FIG. 4 demonstrates npl mutant impairs the IT growth. (Panel A) quantification of nodule numbers after 7 days post-inoculation in open pots. (Panel B) The majority of the infection events in the npl mutant were blocked at the infection chamber stage. (Panels C-J) Phenotypic analysis of plants transformed with empty vector (EV, Panels C and D), ProUBI-NPL-RNAi construct (Panels E and F), epidermis specific promoter (ProEXT1) derived NPL-RNAi construct (Panels G and H), and cortex specific promoter (ProPEP) derived NPL-RNAi construct (Panels I and J). The cell wall was stained with Calcofluor white (white color, in Panels B-J). (K-M″) Phenotypic analysis of plants transformed with NPL promoter derived AtPMEI12. The majority of infections were blocked at the IC stage (Panels K-K″) or during IT formation and initial progression in root hairs (Panels L-L″) and cortex (Panels M-M″). Arrows indicate blocked IC (Panels B, E, G, and K″), Arrowheads indicate the growth of IT either blocked in root hair (Panel L′) or cortex (Panels J and M′). Scale bars indicate 10 μm.

FIG. 5 depicts a proposed model for transcellular IT passage. CLEM analysis for the cell wall modification at the IT penetration sites with antibody LM19 (unesterified pectins, Panel A) and MAC265 (IT matrix, Panel B). (Panel C) using transmission electron microscopy to visualize the cell wall structure labeled by LM19 and (Panel D) is the close-up images of the interesting region marked by a red box in (Panel C). (Panel E) is the proposed model for IT transcellular passage based on our observations. Scale bars indicate 4 μm in (Panels A-C) and 1 μm in D. ID: infection droplet.

FIG. 6 shows immunofluorescence labeling with different cell wall antibodies. 14 days old M. truncatula nodules were embedded in LR Gold and hybridized with the respective antibodies (red). The matrix of infection threads were marked by MAC265 (Panel A); LM25 recognizes hemicellulose xyloglucan (Panel B); LM5 (Panel C) and LM6 (Panel D) recognize different types of RG-I; LM2 (Panel E), LM14 (Panel F), and LM30 (Panel G) all recognize arabinogalactan-proteins (AGPs). DNA was counterstained with DAPI, scale bars indicate 50 μm. Arrows indicate infection thread; CC: colonized cell; NCC: non-colonized cell.

FIG. 7 shows immunofluorescence labeling with LM14 antibody. 14 days old M. truncatula nodules were embedded in LR Gold and hybridized with the LM14 antibody (red). (Panel A) sketch for Medicago nodule; (Panel B) indicates the region corresponding to (Panel B′); (Panel B″) close-up in (Panel B′) marked by a white box. (Panel C) indicates the region corresponding to (Panel C′); (Panel C″) close-up in (Panel C′) marked by a white box; (Panel C″′) fluorescence intensity analysis using a transect as indicated by the line within (Panel C″). DNA was counterstained with DAPI (blue). Arrowheads in (Panel B″) indicate infection thread; arrows in (Panel C″) indicate symbiosome. Scale bars indicate 50 μm in (Panel B′-C′), and 5 μm in C″. IZ: infection zone; FZ: fixation zone; CC: colonized cell; NCC: non-colonized cell.

FIG. 8 shows immunofluorescence labeling with LM6 antibody. 14 days old M. truncatula nodules were embedded in LR Gold and hybridized with the LM6 antibody (red). (Panel A) sketch for Medicago nodule; (Panel B) indicates the region corresponding to (Panel B′); (Panel B″) close-up in (Panel B′) marked by a blue box. (Panel C) indicates the region corresponding to (Panel C′); (Panel C″) close-up in (Panel C′) marked by a magenta box; (Panel C″′) fluorescence intensity analysis using a transect as indicated by the line within (Panel C″). DNA was counterstained with DAPI (blue), scale bars indicate 20 μm. IZ: infection zone; FZ: fixation zone; CC: colonized cell; NCC: non-colonized cell; IT: infection thread.

FIG. 9 shows CLEM analysis for root hair infection events. A correlative light-electron microscopy (CLEM) protocol was established (Panel A); and was used to visualize the cell wall of the IC (Panels B-C) and the IT in root hairs (Panel D).

FIG. 10 shows expression profile of SyPME and analysis of SyPME expression domains (Panel A) Expression patterns of NPL and SyPME are highly correlated (0.9883) as exemplified upon inoculation of roots with a Nod Factor (NF)-deficient S. meliloti nodABC strain (nodABC), WT S. meliloti and isolated NFs at different time points. Data were retrieved from Breakspear et al., 2014. Dpi: days post inoculation; h: hours. (Panel B) Schematic overview of the spatial expression of SyPME and different pectate lyases including NPL in different zones of an indeterminate Medicago nodule. Original data were retrieved from Roux et al., 2014. FI: nodule meristematic zone; zIId: distal of infection zone; zIIp: proximal of infection zone; IZ: interzone; ZIII: nitrogen-fixation zone. (C-F) Spatial analysis of SyPME transcript accumulations by in situ hybridization using a SyPME antisense (Panels C-D) and sense (control) probe (Panels E-F) on 14 days old Medicago nodules. Magenta precipitates indicate presence of SyPME mRNA. (Panels F-H) Promoter GUS (blue) analysis for ProSyPME (Panels G-H) and ProNPL (I) on 14 days old transformed M. truncatula nodules counterstained with Toluidine Blue (purple). Scale bars indicate 50 μm.

FIG. 11 shows localization analysis of SyPME and NPL during primary infection. Images were taken from transformed plants at 7 days post inoculation. Infection chamber (Panels A-A″ and C-C″), growing infection thread (Panels B-B″ and D-D″) and infection threads passage (Panels E-F″). Green signal indicates SyPME in (Panels A-B″ and E-E″), and NPL in (Panels C-D″ and F-F″); red indicates S. meliloti; scale bar indicate 10 μm.

FIG. 12 depicts the gene structure and phenotype of sypme. (Panel A) Schematic representation of the SyPME gene structure and mapped Tnt1 transposon insertion site. UTR, untranslated region. (Panel B) Infection chambers (ICs) and infection threads (ITs) were scored at 10 days after inoculation, with n=10 root systems for each genotype. The IC and IT morphology were visualized by Calcofluor-white staining (White color) in R108 (Panels C-D) and sypme mutants (Panels E-F). Magenta indicates S. meliloti. Scale bars indicate 10 μm.

FIG. 13 depicts a future transcellular passage site, as defined by the cytoplasmatic column formed ahead of the IT. TEM micrograph from a 14 days old nodule with the cell wall colored in yellow, the nucleus outlined in blue, and the cytoplasmic column connecting the infection thread (IT) to the cell wall outlined in red. IT: infection thread; R: rhizobia. Scale bars indicate 2 μm.

### BRIEF DESCRIPTION OF THE SEQUENCES

SEQ ID NO: 1 is a promoter sequence of the M. truncatula SyPME gene, Medtr4g087980.

SEQ ID NO: 2 is a nucleic acid sequence encoding the M. truncatula SyPME protein, Medtr4g087980.

SEQ ID NO: 3 is the amino acid sequence of the M. truncatula SyPME protein, encoded by SEQ ID NO: 2, Medtr4g087980.

SEQ ID NO: 4 is a nucleic acid sequence encoding the M. truncatula SYMREM1 protein.

SEQ ID NO: 5 is the amino acid sequence of the M. truncatula SYMREM1 protein, encoded by SEQ ID NO: 4.

SEQ ID NO: 6 is a nucleic acid genomic sequence encoding the M. truncatula SyPME protein.

### DETAILED DESCRIPTION

Intracellular colonization of host cells by symbionts represents a mutualistic association that occurs between a host plant and soil-borne bacteria or fungi. For example, legumes are known to form a symbiotic relationship with nitrogen-fixing rhizobia, generally referred to as root nodule symbiosis (RNS). While the symbiotic bacteria are initially taken up by morphologically adapted root hairs, rhizobia persistently progress within a membrane-confined infection thread through several root cortical tissues and later nodular cell layers. Throughout this transcellular passage, rhizobia have to repeatedly pass host plasma membranes and cell walls (CW). Furthermore, spatio-temporally confined cell wall remodeling is required to initiate and maintain infection thread (IT) growth, transcellular passage of ITs, as well as bacterial release. This cell wall remodeling is controlled, in part, by the concerted action of a symbiosis-specific pectin methyl esterase (i.e. SyPME; SEQ ID NO: 3) and the pectate lyase NPL at the infection thread and transcellular passage sites, which allows successful intracellular progression of ITs through the entire root cortical tissue. As such, the SyPME promoter (i.e. SEQ ID NO: 1) provides beneficial gene regulatory activity for expression in plant species. For example, the present disclosure demonstrates that the SyPME promoter provides expression within the cortex of nodule primordia, and within a confined expression domain limited to the infection zone II in young and mature nodules. Accordingly, such gene regulatory activity allows for the expression of genes in a symbiosis-dependent and spatially confined manner, e.g. spatial-temporal expression of genes required for effective colonization of engineered nodules and/or nodule-like structures.

The present disclosure, therefore, provides polynucleotide molecules having beneficial gene regulatory activity from plant species. The design, construction, and use of these polynucleotide molecules are provided by the invention. The nucleotide sequences of these polynucleotide molecules are provided herein, e.g. SEQ ID NO: 1. These polynucleotide molecules are, for instance, capable of affecting the expression of an operably linked transcribable polynucleotide molecule in plant tissues, and therefore selectively regulating gene expression, or activity of an encoded gene product, in transgenic plants. The present invention also provides methods of modifying, producing, and using the same. The invention also provides compositions, transformed host cells, transgenic plants, and seeds containing the promoters and/or other disclosed nucleotide sequences, and methods for preparing and using the same.

The following definitions and methods are provided to better define the present invention and to guide those of ordinary skill in the art in the practice of the present invention. Unless otherwise noted, terms are to be understood according to conventional usage by those of ordinary skill in the relevant art.

### Symbiotic Bacteria

The present invention provides DNA molecules having gene regulatory activity. For example, DNA molecules comprising a sequence with at least 85 percent sequence identity to SEQ ID NO: 1 operably linked to a transcribable polynucleotide molecule may be expressed during symbiotic infection by rhizobia. Rhizobia are bacteria found in soil that infect the roots of legumes and colonize root nodules which are involved in nitrogen utilization. As used herein, “rhizobia” refers to any diazotrophic bacteria that fix atmospheric nitrogen inside plants' roots. Symbiotic bacteria can be used with plants comprising the recombinant DNA molecules described herein. Symbiotic bacteria useful with the disclosed plants include, but are not limited to, Mesorhizobium loti, Sinorhizobium meliloti, Sinorhizobium fredii, and Bradyrhizobium sp.

### DNA Molecules

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a DNA molecule, or fragment thereof, disclosed in the present invention. For example, PCR (polymerase chain reaction) technology can be used to amplify a particular starting DNA molecule and/or to produce variants of the original molecule. DNA molecules, or fragment thereof, can also be obtained by other techniques such as by directly synthesizing the fragment by chemical means, as is commonly practiced by using an automated oligonucleotide synthesizer.

As used herein, the term “sequence identity” refers to the extent to which two optimally aligned polynucleotide sequences or two optimally aligned polypeptide sequences are identical. An optimal sequence alignment is created by manually aligning two sequences, e.g. a reference sequence and another sequence, to maximize the number of nucleotide matches in the sequence alignment with appropriate internal nucleotide insertions, deletions, or gaps. As used herein, the term “reference sequence” refers to a sequence provided as the polynucleotide sequences of SEQ ID NO: 1.

As used herein, the term “percent sequence identity” or “percent identity” or “% identity” is the identity fraction times 100. The “identity fraction” for a sequence optimally aligned with a reference sequence is the number of nucleotide matches in the optimal alignment, divided by the total number of nucleotides in the reference sequence, e.g. the total number of nucleotides in the full length of the entire reference sequence. Thus, one embodiment of the invention is a DNA molecule comprising a sequence that when optimally aligned to a reference sequence, provided herein as SEQ ID NO: 1, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments, such sequences may be defined as having gene-regulatory activity.

### Regulatory Elements

A regulatory element is a DNA molecule having gene regulatory activity, i.e. one that has the ability to affect the transcription and/or translation of an operably linked transcribable polynucleotide molecule. The term “gene regulatory activity” thus refers to the ability to affect the expression pattern of an operably linked transcribable polynucleotide molecule by affecting the transcription and/or translation of that operably linked transcribable polynucleotide molecule. As used herein, a transcriptional regulatory sequence may be comprised of operably linked expression elements, such as enhancers, promoters, leaders, and introns. Thus a transcriptional regulatory sequence may be comprised, for instance, of a promoter operably linked 5′ to a leader sequence, which is in turn operably linked 5′ to an intron sequence. Leaders and introns may positively affect transcription of an operably linked transcribable polynucleotide molecule as well as translation of the resulting transcribed RNA. The pre-processed RNA molecule comprises leaders and introns, which may affect the post-transcriptional processing of the transcribed RNA and/or the export of the transcribed RNA molecule from the cell nucleus into the cytoplasm. Following post-transcriptional processing of the transcribed RNA molecule, the leader sequence may be retained as part of the final messenger RNA and may positively affect the translation of the messenger RNA molecule.

Regulatory elements such as promoters, leaders, introns, and transcription termination regions (or 3′ UTRs) are DNA molecules that have gene regulatory activity and play an integral part in the overall expression of genes in living cells. The term “regulatory element” refers to a DNA molecule having gene regulatory activity, i.e. one that has the ability to affect the transcription and/or translation of an operably linked transcribable polynucleotide molecule. Isolated regulatory elements, such as promoters and leaders that function in plants are therefore useful for modifying plant phenotypes through the methods of genetic engineering.

Regulatory elements may be characterized by their expression pattern effects (qualitatively and/or quantitatively), e.g. positive or negative effects and/or constitutive or other effects such as by their temporal, spatial, developmental, tissue, environmental, physiological, pathological, cell cycle, and/or chemically responsive expression pattern, and any combination thereof, as well as by quantitative or qualitative indications. A promoter is useful as a regulatory element for modulating the expression of an operably linked transcribable polynucleotide molecule.

As used herein, a “gene expression pattern” is any pattern of transcription of an operably linked DNA molecule into a transcribed RNA molecule. The transcribed RNA molecule may be translated to produce a protein molecule or may provide an antisense or other regulatory RNA molecule, such as a dsRNA, a tRNA, an rRNA, a miRNA, and the like.

As used herein, the term “protein expression” is any pattern of translation of a transcribed RNA molecule into a protein molecule. Protein expression may be characterized by its temporal, spatial, developmental, or morphological qualities as well as by quantitative or qualitative indications.

As used herein, the term “promoter” refers generally to a DNA molecule that is involved in recognition and binding of RNA polymerase II and other proteins (trans-acting transcription factors) to initiate transcription. A promoter may be initially isolated from the 5′ untranslated region (5′ UTR) of a genomic copy of a gene. Alternately, promoters may be synthetically produced or manipulated DNA molecules. Promoters may also be chimeric, that is a promoter produced through the fusion of two or more heterologous DNA molecules. Promoters useful in practicing the present invention include SEQ ID NO: 1, or fragments or variants thereof. In specific embodiments of the invention, such molecules and any variants or derivatives thereof as described herein, are further defined as comprising gene regulatory activity or promoter activity, i.e., are capable of acting as a promoter in a host cell, such as in a transgenic plant. In still further specific embodiments, a fragment may be defined as exhibiting gene regulatory activity or promoter activity possessed by the starting promoter molecule from which it is derived, or a fragment may comprise a “minimal promoter” which provides a basal level of transcription and is comprised of a TATA box or equivalent sequence for recognition and binding of the RNA polymerase II complex for initiation of transcription.

In one embodiment, fragments are provided of a promoter sequence disclosed herein. Promoter fragments may comprise gene regulatory activity or promoter activity, as described above, and may be useful alone or in combination with other promoters and promoter fragments, such as in constructing chimeric promoters. In specific embodiments, fragments of a promoter are provided comprising at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 500, 750, 1000, 1250, 1500, 1750, or at least about 2000 contiguous nucleotides or bases, or longer, of a polynucleotide molecule having gene regulatory activity or promoter activity disclosed herein. A fragment of a promoter can be a functional fragment having gene regulatory function or activity or promoter function or activity. The recitation of discrete values is understood to include ranges between each value and discrete values between the ranges.

Compositions derived from any of the promoters presented as SEQ ID NO: 1, such as internal or 5′ deletions, for example, can be produced using methods known in the art to improve or alter expression, including by removing elements that have either positive or negative effects on expression; duplicating elements that have positive or negative effects on expression; and/or duplicating or removing elements that have tissue or cell specific effects on expression. Compositions derived from any of the promoters presented as SEQ ID NO: 1 comprised of 3′ deletions in which the TATA box element or equivalent sequence thereof and downstream sequence is removed can be used, for example, to make enhancer elements. Further deletions can be made to remove any elements that have positive or negative; tissue specific; cell specific; or timing specific (such as, but not limited to, circadian rhythms) effects on expression. Any of the promoters presented as SEQ ID NO: 1 and fragments or enhancers derived therefrom can be used to make chimeric transcriptional regulatory element compositions comprised of any of the promoters presented as SEQ ID NO: 1 and the fragments or enhancers derived therefrom operably linked to other enhancers and promoters. The efficacy of the modifications, duplications, or deletions described herein on the desired expression aspects of a particular transgene may be tested empirically in stable and transient plant assays, such as those described in the working examples herein, so as to validate the results, which may vary depending upon the changes made and the goal of the change in the starting molecule.

As used herein, the term “leader” refers to a DNA molecule isolated from the untranslated 5′ region (5′ UTR) of a genomic copy of a gene and defined generally as a nucleotide segment between the transcription start site (TSS) and the protein coding sequence start site. Alternately, leaders may be synthetically produced or manipulated DNA elements. A leader can be used as a 5′ regulatory element for modulating expression of an operably linked transcribable polynucleotide molecule. Leader molecules may be used with a heterologous promoter or with their native promoter. Promoter molecules of the present invention may thus be operably linked to their native leader or may be operably linked to a heterologous leader. Leaders known in the art may be useful in practicing the present invention. The leader sequences (5′ UTR) may be comprised of regulatory elements or may adopt secondary structures that can have an effect on transcription or translation of a transgene. Leader sequences known in the art can be used in accordance with the invention to make chimeric regulatory elements that affect transcription or translation of a transgene. In addition, leader sequences can be used to make chimeric leader sequences that affect transcription or translation of a transgene.

The introduction of a foreign gene into a new plant host does not always result in a high expression of the incoming gene. Furthermore, if dealing with complex traits, it is sometimes necessary to modulate several genes with spatially or temporarily different expression pattern. Introns can principally provide such modulation. However multiple use of the same intron in one plant has shown to exhibit disadvantages. In those cases, it is necessary to have a collection of basic control elements for the construction of appropriate recombinant DNA elements.

In accordance with the invention a promoter or promoter fragment may be analyzed for the presence of known promoter elements, i.e. DNA sequence characteristics, such as a TATA-box and other known transcription factor binding site motifs. Identification of such known promoter elements may be used by one of skill in the art to design variants of the promoter having a similar expression pattern to the original promoter.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide sequence. Unlike promoters, enhancer elements do not usually include a transcription start site (TSS) or TATA box or equivalent sequence. A promoter may naturally comprise one or more enhancer elements that affect the transcription of an operably linked polynucleotide sequence. An isolated enhancer element may also be fused to a promoter to produce a chimeric promoter cis-element, which confers an aspect of the overall modulation of gene expression. A promoter or promoter fragment may comprise one or more enhancer elements that affect the transcription of operably linked genes. Many promoter enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element may function to bind transcription factors that regulate transcription. Some enhancer elements bind more than one transcription factor, and transcription factors may interact with different affinities with more than one enhancer domain. Enhancer elements can be identified by a number of techniques, including deletion analysis, i.e. deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis using known cis-element motifs or enhancer elements as a target sequence or target motif with conventional DNA sequence comparison methods, such as BLAST. The fine structure of an enhancer domain can be further studied by mutagenesis (or substitution) of one or more nucleotides or by other conventional methods. Enhancer elements can be obtained by chemical synthesis or by isolation from regulatory elements that include such elements, and they can be synthesized with additional flanking nucleotides that contain useful restriction enzyme sites to facilitate subsequence manipulation. Thus, the design, construction, and use of enhancer elements according to the methods disclosed herein for modulating the expression of operably linked transcribable polynucleotide molecules are encompassed by the present invention.

In plants, the inclusion of some introns in gene constructs leads to increased mRNA and protein accumulation relative to constructs lacking the intron.

This effect has been termed “intron mediated enhancement” (IME) of gene expression (Mascarenhas et al., (1990) Plant Mol. Biol. 15:913-920). Introns known to stimulate expression in plants have been identified in maize genes (e.g. tubA1, Adh1, Sh1, Ubi1 (Jeon et al. (2000) Plant Physiol. 123:1005-1014; Callis et al. (1987) Genes Dev. 1:1183-1200; Vasil et al. (1989) Plant Physiol. 91:1575-1579; Christiansen et al. (1992) Plant Mol. Biol. 18:675-689) and in rice genes (e.g. salt, tpi: McElroy et al., Plant Cell 2:163-171 (1990); Xu et al., Plant Physiol. 106:459-467 (1994)). Similarly, introns from dicotyledonous plant genes like those from petunia (e.g. rbcS), potato (e.g. st-1s1) and from Arabidopsis thaliana (e.g. ubq3 and pat1) have been found to elevate gene expression rates (Dean et al. (1989) Plant Cell 1:201-208; Leon et al. (1991) Plant Physiol. 95:968-972; Norris et al. (1993) Plant Mol Biol 21:895-906; Rose and Last (1997) Plant J. 11:455-464). It has been shown that deletions or mutations within the splice sites of an intron reduce gene expression, indicating that splicing might be needed for IME (Mascarenhas et al. (1990) Plant Mol Biol. 15:913-920; Clancy and Hannah (2002) Plant Physiol. 130:918-929). However, that splicing per se is not required for a certain IME in dicotyledonous plants has been shown by point mutations within the splice sites of the pat1 gene from A. thaliana (Rose and Beliakoff (2000) Plant Physiol. 122:535-542).

Enhancement of gene expression by introns is not a general phenomenon because some intron insertions into recombinant expression cassettes fail to enhance expression (e.g. introns from dicot genes (rbcS gene from pea, phaseolin gene from bean and the st/s-1 gene from Solanum tuberosum) and introns from maize genes (adh1 gene the ninth intron, hsp81 gene the first intron)) (Chee et al. (1986) Gene 41:47-57; Kuhlemeier et al. (1988) Mol Gen Genet 212:405-411; Mascarenhas et al. (1990) Plant Mol. Biol. 15:913-920; Sinibaldi and Mettler (1992) In W E Cohn, K Moldave, eds, Progress in Nucleic Acid Research and Molecular Biology, Vol 42. Academic Press, New York, pp 229-257; Vancanneyt et al. 1990 Mol. Gen. Genet. 220:245-250). Therefore, not each intron can be employed in order to manipulate the gene expression level of non-endogenous genes or endogenous genes in transgenic plants. What characteristics or specific sequence features must be present in an intron sequence in order to enhance the expression rate of a given gene is not known in the prior art and therefore from the prior art it is not possible to predict whether a given plant intron, when used heterologously, will cause enhancement of expression at the DNA level or at the transcript level (IME).

As used herein, the term “chimeric” refers to a single DNA molecule produced by fusing a first DNA molecule to a second DNA molecule, where neither first nor second DNA molecule would normally be found in that configuration, i.e. fused to the other. The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments; an example would be the fusion of a promoter to an enhancer element. Thus, the design, construction, and use of chimeric promoters according to the methods disclosed herein for modulating the expression of operably linked transcribable polynucleotide molecules are encompassed by the present invention.

As used herein, the term “variant” refers to a second DNA molecule that is in composition similar, but not identical to, a first DNA molecule and yet the second DNA molecule still maintains the general functionality, i.e. same or similar expression pattern, of the first DNA molecule. A variant may be a shorter or truncated version of the first DNA molecule and/or an altered version of the sequence of the first DNA molecule, such as one with different restriction enzyme sites and/or internal deletions, substitutions, and/or insertions. A “variant” can also encompass a regulatory element having a nucleotide sequence comprising a substitution, deletion and/or insertion of one or more nucleotides of a reference sequence, wherein the derivative regulatory element has more or less or equivalent transcriptional or translational activity than the corresponding parent regulatory molecule. The regulatory element “variants” will also encompass variants arising from mutations that naturally occur in bacterial and plant cell transformation. In the present invention, a polynucleotide sequence provided as SEQ ID NO: 1 may be used to create variants that are in composition similar, but not identical to, the polynucleotide sequence of the original regulatory element, while still maintaining the general functionality, i.e. same or similar expression pattern, of the original regulatory element. Production of such variants of the present invention is well within the ordinary skill of the art in light of the disclosure and is encompassed within the scope of the present invention. Chimeric regulatory element “variants” comprise the same constituent elements as a reference sequence but the constituent elements comprising the chimeric regulatory element may be operatively linked by various methods known in the art such as, restriction enzyme digestion and ligation, ligation independent cloning, modular assembly of PCR products during amplification, or direct chemical synthesis of the regulatory element as well as other methods known in the art. The resulting chimeric regulatory element “variant” can be comprised of the same, or variants of the same, constituent elements of the reference sequence but differ in the sequence or sequences that comprise the linking sequence or sequences which allow the constituent parts to be operatively linked. In the present invention, a polynucleotide sequence provided as SEQ ID NO: 1 provide a reference sequence wherein the constituent elements that comprise the reference sequence may be joined by methods known in the art and may comprise substitutions, deletions and/or insertions of one or more nucleotides or mutations that naturally occur in bacterial and plant cell transformation.

### Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. As used herein, the term “vector” means any recombinant polynucleotide construct that may be used for the purpose of transformation, i.e. the introduction of heterologous DNA into a host cell. The term includes an expression cassette isolated from any of the aforementioned molecules.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. A leader, for example, is operably linked to coding sequence when it is capable of serving as a leader for the polypeptide encoded by the coding sequence.

The constructs of the present invention may be provided, in one embodiment, as double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the A. tumefaciens cells, permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known to those skilled in the art of plant transformation can function in the present invention.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art, see, for example, Molecular Cloning: A Laboratory Manual, 3^{rd} edition Volumes 1, 2, and 3 (2000) J. Sambrook, D. W. Russell, and N. Irwin, Cold Spring Harbor Laboratory Press. Methods for making recombinant vectors particularly suited to plant transformation include, without limitation, those described in U.S. Pat. Nos. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, (1988) and Glick, et al., Methods in Plant Molecular Biology and Biotechnology, CRC Press, Boca Raton, FL. (1993)). Typical vectors useful for expression of nucleic acids in higher plants are well known in the art and include vectors derived from the tumor-inducing (Ti) plasmid of Agrobacterium tumefaciens (Rogers, et al., Methods in Enzymology 153: 253-277 (1987)). Other recombinant vectors useful for plant transformation, including the pCaMVCN transfer control vector, have also been described in the scientific literature (see, for example, Fromm, et al., Proc. Natl. Acad. Sci. USA 82: 5824-5828 (1985)).

Various regulatory elements may be included in a construct including any of those provided herein. Any such regulatory elements may be provided in combination with other regulatory elements. Such combinations can be designed or modified to produce desirable regulatory features. In one embodiment, constructs of the present invention comprise at least one regulatory element operably linked to a transcribable polynucleotide molecule operably linked to a 3′ UTR.

Constructs of the present invention may include any promoter or leader provided herein or known in the art. For example, a promoter of the present invention may be operably linked to a heterologous non-translated 5′ leader such as one derived from a heat shock protein gene (see, for example, U.S. Pat. Nos. 5,659,122 and 5,362,865). Alternatively, a leader of the present invention may be operably linked to a heterologous promoter such as the Cauliflower Mosaic Virus 35S transcript promoter (see, U.S. Pat. No. 5,352,605).

As used herein, the term “intron” refers to a DNA molecule that may be isolated or identified from the genomic copy of a gene and may be defined generally as a region spliced out during mRNA processing prior to translation. Alternately, an intron may be a synthetically produced or manipulated DNA element. An intron may contain enhancer elements that effect the transcription of operably linked genes. An intron may be used as a regulatory element for modulating expression of an operably linked transcribable polynucleotide molecule. A DNA construct may comprise an intron, and the intron may or may not be heterologous with respect to the transcribable polynucleotide molecule sequence. Examples of introns in the art include the rice actin intron (U.S. Pat. No. 5,641,876) and the corn HSP70 intron (U.S. Pat. No. 5,859,347). Further, when modifying intron/exon boundary sequences, it may be preferable to avoid using the nucleotide sequence AT or the nucleotide A just prior to the 5′ end of the splice site (GT) and the nucleotide G or the nucleotide sequence TG, respectively just after 3′ end of the splice site (AG) to eliminate the potential of unwanted start codons from being formed during processing of the messenger RNA into the final transcript. The sequence around the 5′ or 3′ end splice junction sites of the intron can thus be modified in this manner.

As used herein, the term “3′ transcription termination molecule” or “3′ UTR” refers to a DNA molecule that is used during transcription to produce the 3′ untranslated region (3′ UTR) of an mRNA molecule. The 3′ untranslated region of an mRNA molecule may be generated by specific cleavage and 3′ polyadenylation, a.k.a. polyA tail. A 3′ UTR may be operably linked to and located downstream of a transcribable polynucleotide molecule and may include polynucleotides that provide a polyadenylation signal and other regulatory signals capable of affecting transcription, mRNA processing, or gene expression. PolyA tails are thought to function in mRNA stability and in initiation of translation. Examples of 3′ transcription termination molecules in the art are the nopaline synthase 3′ region (see, Fraley, et al., Proc. Natl. Acad. Sci. USA, 80: 4803-4807 (1983)); wheat hsp17 3′ region; pea rubisco small subunit 3′ region; cotton E6 3′ region (U.S. Pat. No. 6,096,950); 3′ regions disclosed in WO0011200A2; and the coixin 3′ UTR (U.S. Pat. No. 6,635,806).

### Transcribable Polynucleotide Molecules

As used herein, the term “transcribable polynucleotide molecule” refers to any DNA molecule capable of being transcribed into a RNA molecule, including, but not limited to, those having protein coding sequences and those producing RNA molecules having sequences useful for gene suppression. A “transgene” refers to a transcribable polynucleotide molecule heterologous to a host cell at least with respect to its location in the genome and/or a transcribable polynucleotide molecule artificially incorporated into a host cell's genome in the current or any prior generation of the cell.

A promoter of the present invention may be operably linked to a transcribable polynucleotide molecule that is heterologous with respect to the promoter molecule. As used herein, the term “heterologous” refers to the combination of two or more polynucleotide molecules when such a combination is not normally found in nature. For example, the two molecules may be derived from different species and/or the two molecules may be derived from different genes, e.g. different genes from the same species or the same genes from different species. Additionally, the two molecules may be derived from isolated locations in the same gene, wherein such a combination of molecules is not normally found in nature. A promoter is thus heterologous with respect to an operably linked transcribable polynucleotide molecule if such a combination is not normally found in nature, i.e. that transcribable polynucleotide molecule is not naturally occurring operably linked in combination with that promoter molecule.

As used herein, the term “overexpression” refers to an increased expression level of a transcribable polynucleotide molecule or a protein in a plant, plant cell, or plant tissue, compared to expression in a wild-type plant, cell, or tissue, at any developmental or temporal stage for the gene. Overexpression can take place in plant cells normally lacking expression of a transcribable polynucleotide molecule of interest. Overexpression can also occur in plant cells where endogenous expression of a transcribable polynucleotide molecule or functionally equivalent molecules normally occurs, but such endogenous expression is at a lower level compared to the overexpression. Overexpression thus results in a greater than endogenous production, or “overproduction” of the polypeptide in the plant, cell, or tissue.

In certain embodiments, the expression or overexpression of a transcribable polynucleotide molecule as disclosed herein can affect an enhanced trait or altered phenotype directly or indirectly. In some cases it may do so, for example, by promoting effective colonization by symbiotic bacteria. In certain exemplary embodiments, the protein produced from the transcribable polynucleotide molecule can modulate cell wall texture in cells colonized by bacteria.

The transcribable polynucleotide molecule may generally be any DNA molecule for which expression of a RNA transcript is desired. Such expression of an RNA transcript may result in translation of the resulting mRNA molecule and thus protein expression. Alternatively, for example, a transcribable polynucleotide molecule may be designed to ultimately cause decreased expression of a specific gene or protein. In one embodiment, this may be accomplished by using a transcribable polynucleotide molecule that is oriented in the antisense direction. One of ordinary skill in the art is familiar with using such antisense technology. Briefly, as the antisense transcribable polynucleotide molecule is transcribed, the RNA product hybridizes to and sequesters a complementary RNA molecule inside the cell. This duplex RNA molecule cannot be translated into a protein by the cell's translational machinery and is degraded in the cell. Any gene may be negatively regulated in this manner.

Thus, one embodiment of the invention is a regulatory element of the present invention, such as those provided as SEQ ID NO: 1, operably linked to a transcribable polynucleotide molecule so as to modulate transcription of the transcribable polynucleotide molecule at a desired level or in a desired pattern when the construct is integrated in the genome of a plant cell. In one embodiment, the transcribable polynucleotide molecule comprises a protein-coding region of a gene, and the promoter affects the transcription of an RNA molecule that is translated and expressed as a protein product. In another embodiment of the invention, the transcribable polynucleotide molecule comprises a sequence encoding a protein, wherein said protein comprises an amino acid sequence having at least 85%, or 90%, or 95%, or 98% or 99%, or about 100% amino acid sequence identity to any of SEQ ID NOs: 3 and 5. In particular embodiments, such sequences may be defined as having the activity of the reference sequence, for example the activity of any of SEQ ID NOs: 3 and 5.

In another embodiment, the transcribable polynucleotide molecule comprises an antisense region of a gene, and the promoter affects the transcription of an antisense RNA molecule, double stranded RNA, or other similar inhibitory RNA molecule in order to inhibit expression of a specific RNA molecule of interest in a target host cell.

### Genes of Agronomic Interest

Transcribable polynucleotide molecules may be genes of agronomic interest. As used herein, the term “gene of agronomic interest” refers to a transcribable polynucleotide molecule that when expressed in a particular plant tissue, cell, or cell type confers a desirable characteristic, such as associated with plant morphology, physiology, growth, development, yield, product, nutritional profile, disease, or pest resistance, and/or environmental or chemical tolerance. Genes of agronomic interest include, but are not limited to, those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent such as an antisense or RNAi molecule targeting a particular gene for suppression. In some embodiments, genes of agronomic interest include, but are not limited to, those encoding pectate lyases (e.g. NPL) and pectin methylesterases (e.g. SyPME) to modulate cell wall texture in cells colonized by bacteria; genes encoding proteins with scaffolding functions (e.g. SYMREM1); genes encoding for high affinity cytochrome oxidases (e.g. cbb3-type oxidase) to increase cellular respiration under low free oxygen conditions; and genes encoding leghemoglobins to create a hypoxic environment within an engineered nodule-like structure. In particular embodiments, a gene of agronomic interest may comprise any of SEQ ID NOs: 2, 4, and 6; or may comprise a polynucleotide segment encoding a protein, wherein said protein comprises the amino acid sequence of any of SEQ ID NOs: 3 and 5. The product of a gene of agronomic interest may act within the plant in order to cause an effect upon the plant physiology or metabolism or may act to promote the formation and development of nodule-like structures on the plant.

Alternatively, a gene of agronomic interest can affect the above mentioned plant characteristic or phenotype by encoding a RNA molecule that causes the targeted modulation of gene expression of an endogenous gene, for example via antisense (see e.g. U.S. Pat. No. 5,107,065); inhibitory RNA (“RNAi”, including modulation of gene expression via miRNA-, siRNA-, trans-acting siRNA-, and phased sRNA-mediated mechanisms, e.g. as described in published applications US 2006/0200878 and US 2008/0066206, and in U.S. patent application Ser. No. 11/974,469); or cosuppression-mediated mechanisms. The RNA could also be a catalytic RNA molecule (e.g. a ribozyme or a riboswitch; see e.g. US 2006/0200878) engineered to cleave a desired endogenous mRNA product. Thus, any transcribable polynucleotide molecule that encodes a transcribed RNA molecule that affects an agronomically important phenotype or morphology change of interest may be useful for the practice of the present invention. Methods are known in the art for constructing and introducing constructs into a cell in such a manner that the transcribable polynucleotide molecule is transcribed into a molecule that is capable of causing gene suppression. For example, posttranscriptional gene suppression using a construct with an anti-sense oriented transcribable polynucleotide molecule to regulate gene expression in plant cells is disclosed in U.S. Pat. Nos. 5,107,065 and 5,759,829, and posttranscriptional gene suppression using a construct with a sense-oriented transcribable polynucleotide molecule to regulate gene expression in plants is disclosed in U.S. Pat. Nos. 5,283,184 and 5,231,020. Expression of a transcribable polynucleotide in a plant cell can also be used to suppress plant pests feeding on the plant cell, for example, compositions isolated from coleopteran pests (U.S. Patent Publication No. US20070124836) and compositions isolated from nematode pests (U.S. Patent Publication No. US20070250947). Plant pests include, but are not limited to arthropod pests, nematode pests, and fungal or microbial pests. Exemplary transcribable polynucleotide molecules for incorporation into constructs of the present invention include, for example, DNA molecules or genes from a species other than the target species or genes that originate with or are present in the same species, but are incorporated into recipient cells by genetic engineering methods rather than classical reproduction or breeding techniques. The type of polynucleotide molecule can include, but is not limited to, a polynucleotide molecule that is already present in the plant cell, a polynucleotide molecule from another plant, a polynucleotide molecule from a different organism, or a polynucleotide molecule generated externally, such as a polynucleotide molecule containing an antisense message of a gene, or a polynucleotide molecule encoding an artificial, synthetic, or otherwise modified version of a transgene.

### Selectable Markers

As used herein the term “marker” refers to any transcribable polynucleotide molecule whose expression, or lack thereof, can be screened for or scored in some way. Marker genes for use in the practice of the present invention include, but are not limited to transcribable polynucleotide molecules encoding ß-glucuronidase (GUS described in U.S. Pat. No. 5,599,670), green fluorescent protein and variants thereof (GFP described in U.S. Pat. Nos. 5,491,084 and 6,146,826), proteins that confer antibiotic resistance, or proteins that confer herbicide tolerance.

Included within the term “selectable markers” are also genes which encode a secretable marker whose secretion can be detected as a means of identifying or selecting for transformed cells. Examples include markers that encode a secretable antigen that can be identified by antibody interaction, or even secretable enzymes which can be detected catalytically. Selectable secreted marker proteins fall into a number of classes, including small, diffusible proteins which are detectable, (e.g. by ELISA), small active enzymes which are detectable in extracellular solution (e.g, alpha-amylase, beta-lactamase, phosphinothricin transferase), or proteins which are inserted or trapped in the cell wall (such as proteins which include a leader sequence such as that found in the expression unit of extension or tobacco pathogenesis related proteins also known as tobacco PR-S). Other possible selectable marker genes will be apparent to those of skill in the art and are encompassed by the present invention.

### Cell Transformation

The invention is also directed to a method of producing transformed cells and plants which comprise a promoter operably linked to a transcribable polynucleotide molecule.

The term “transformation” refers to the introduction of nucleic acid into a recipient host. As used herein, the term “host” refers to bacteria, fungi, or plant, including any cells, tissue, organs, or progeny of the bacteria, fungi, or plant. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign polynucleotide molecule, such as a construct, has been introduced. The introduced polynucleotide molecule may be integrated into the genomic DNA of the recipient cell, tissue, organ, or organism such that the introduced polynucleotide molecule is inherited by subsequent progeny. A “transgenic” or “transformed” cell or organism also includes progeny of the cell or organism and progeny produced from a breeding program employing such a transgenic organism as a parent in a cross and exhibiting an altered phenotype resulting from the presence of a foreign polynucleotide molecule. The term “transgenic” refers to a bacteria, fungi, or plant containing one or more heterologous polynucleic acid molecules.

There are many methods for introducing polynucleic acid molecules into plant cells. The method generally comprises the steps of selecting a suitable host cell, transforming the host cell with a recombinant vector, and obtaining the transformed host cell. Suitable methods include bacterial infection (e.g. Agrobacterium), binary bacterial artificial chromosome vectors, direct delivery of DNA (e.g. via PEG-mediated transformation, desiccation/inhibition-mediated DNA uptake, electroporation, agitation with silicon carbide fibers, and acceleration of DNA coated particles, etc. (reviewed in Potrykus, et al., Ann. Rev. Plant Physiol. Plant Mol. Biol. 42: 205 (1991)).

Technology for introduction of a DNA molecule into cells is well known to those of skill in the art. Methods and materials for transforming plant cells by introducing a plant DNA construct into a plant genome in the practice of this invention can include any of the well-known and demonstrated methods. Any transformation methods may be utilized to transform a host cell with one or more promoters and/or constructs of the present. Host cells may be any cell or organism such as a plant cell, algae cell, algae, fungal cell, fungi, bacterial cell, or insect cell. Preferred hosts and transformed cells include cells from: plants, Aspergillus, yeasts, insects, bacteria, and algae.

Regenerated transgenic plants can be self-pollinated to provide homozygous transgenic plants. Alternatively, pollen obtained from the regenerated transgenic plants may be crossed with non-transgenic plants, preferably inbred lines of agronomically important species. Descriptions of breeding methods that are commonly used for different traits and crops can be found in one of several reference books, see, for example, Allard, Principles of Plant Breeding, John Wiley & Sons, NY, U. of CA, Davis, CA, 50-98 (1960); Simmonds, Principles of crop improvement, Longman, Inc., NY, 369-399 (1979); Sneep and Hendriksen, Plant breeding perspectives, Wageningen (ed), Center for Agricultural Publishing and Documentation (1979); Fehr, Soybeans: Improvement, Production and Uses, 2nd Edition, Monograph, 16:249 (1987); Fehr, Principles of variety development, Theory and Technique, (Vol. 1) and Crop Species Soybean (Vol 2), Iowa State Univ., Macmillan Pub. Co., NY, 360-376 (1987). Conversely, pollen from non-transgenic plants may be used to pollinate the regenerated transgenic plants.

The transformed plants may be analyzed for the presence of the genes of interest and the expression level and/or profile conferred by the regulatory elements of the present invention. Those of skill in the art are aware of the numerous methods available for the analysis of transformed plants. For example, methods for plant analysis include, but are not limited to Southern blots or northern blots, PCR-based approaches, biochemical analyses, phenotypic screening methods, field evaluations, and immunodiagnostic assays. The expression of a transcribable polynucleotide molecule can be measured using TaqMan® (Applied Biosystems, Foster City, CA) reagents and methods as described by the manufacturer and PCR cycle times determined using the TaqMan® Testing Matrix. Alternatively, the Invader® (Third Wave Technologies, Madison, WI) reagents and methods as described by the manufacturer can be used transgene expression.

The seeds of the plants of this invention can be harvested from fertile transgenic plants and be used to grow progeny generations of transformed plants of this invention including hybrid plant lines comprising the construct of this invention and expressing a gene of agronomic interest.

The present invention also provides for parts of the plants of the present invention. Plant parts, without limitation, include leaves, stems, roots, tubers, seeds, endosperm, ovule, and pollen. The invention also includes and provides transformed plant cells which comprise a nucleic acid molecule of the present invention.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. The progeny from these plants are evaluated, among other things, for gene expression. The gene expression may be detected by several common methods such as western blotting, northern blotting, immunoprecipitation, and ELISA.

A transgenic plant, plant cell, plant part, or progeny thereof as described herein can be selected from the group of an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn, cowpea, clover, cotton, a cucurbit, cucumber, Douglas fir, eggplant, eucalyptus, flax, garlic, forage legumes, grape, hemp, hops, indigo, leek, legume trees, lentil, lettuce, Loblolly pine, lotus, lupin, millets, melons, Medicago spp., nut, oat, olive, onion, ornamental, palm, pasture grass, pea, peach, peanut, pepper, pigeon pea, pine, potato, poplar, pumpkin, pulses, Radiata pine, radish, rapeseed, raspberry, rice, rootstocks, rye, red currant, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, walnut, watermelon, wheat, or yam.

### Commodity Products

The present invention provides a commodity product comprising DNA molecules according to the invention. As used herein, a “commodity product” refers to any composition or product which is comprised of material derived from a plant, seed, plant cell or plant part comprising a DNA molecule of the invention. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animals consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the invention can thus be used to manufacture any commodity product typically acquired from plants or parts thereof.

Having now generally described the invention, the same will be more readily understood through reference to the following examples which are provided by way of illustration, and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1: Analysis of Cell Wall Structures Surrounding Infection Threads

To study cell wall structure and composition during rhizobial infections and transcellular IT passage, a set of immuno-labelling experiments on indeterminate M. truncatula nodules was run using a series of selected antibodies against different cell wall constituents (Table 1). In brief, seedlings were directly grown in open pots (mixture of 1:1 quarzsand:vermiculite mixture, 2 plants/pot) after seed germination. Plants were watered with liquid Fahrädeus medium (without nitrate, 30 ml/pot) and tap water (30 ml/pot) once a week, individually. The plants were then inoculated with S. meliloti (OD600=0.003) 7 days after transfer (20 ml/pot). Another 10 days later, plants were harvested for the quantification of infection structures.

TABLE 1
List of cell wall antibodies used to study cell
wall structure and composition during rhizobial
infections and transcellular IT passage.

| Antibody name | Epitope |
|---|---|
| MAC265 | Infection thread matrix glycoprotein |
| LM5 | Pectic polysaccharide (beta 1,4-galactan) |
| LM6 | Pectic polysaccharide (alpha-1,5-arabinan) |
| LM19 | Unesterified homogalacturonan |
| LM20 | Methyl esterified homogalacturonan |
| 2F4 | ‘egg box” dimer conformation of homogalacturonan |
| LM2 | Arabinogalactan-protein |
| LM14 | Arabinogalactan-protein |
| LM30 | Arabinogalactan-protein |

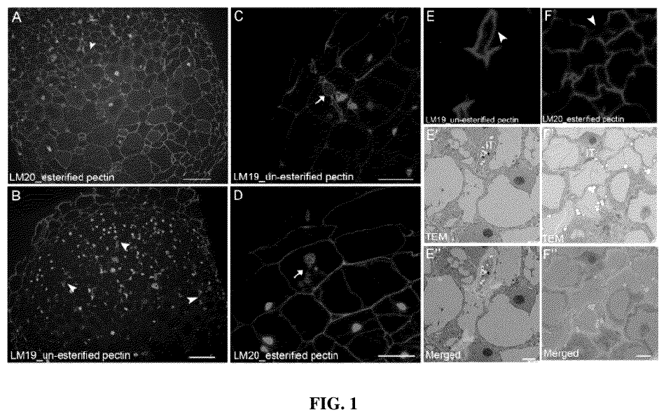

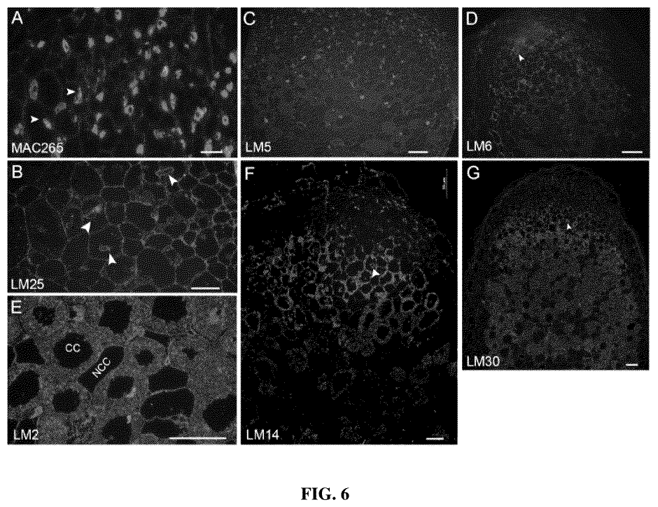

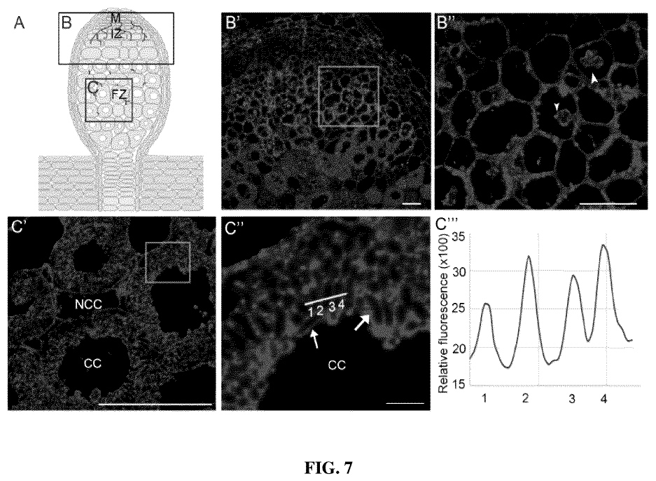

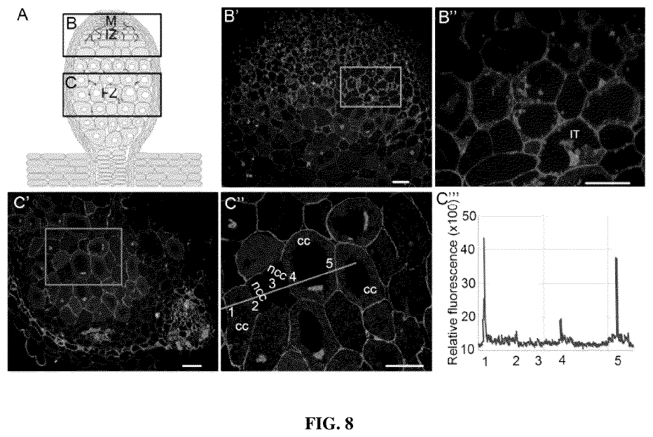

To identify ITs, IT matrix glycoproteins were first labelled using the MAC265 antibody (VandenBosch et al., 1989). As expected, nodular ITs were specifically labelled by this approach while the peripheral cell wall of nodule cortex cells did not show any fluorescent signal (FIG. 6, Panel A). This was different when targeting xyloglucan as the most abundant hemicellulose by LM25. Here, ubiquitous labelling was found for the cell periphery of all nodule cells including ITs (FIG. 6, Panel B). In contrast, different arabinogalactan proteins (labelled by LM2, LM14, LM30) that have been reported to serve functions during plant-microbe interactions were found to specifically accumulate within the infection zone and around symbiosomes (FIG. 6, Panels E-G and FIG. 7). Next, the presence of different pectins were addressed including rhamnogalacturonans I (RG-I), homogalacturonans (HG), rhamnogalacturonan II (RG-II), and xylogalacturonan. The linear (1-4)-β-D-galactan (recognized by LM5), an epitope of RG-I, was barely detectable inside the Medicago nodule sections (FIG. 6, Panel C). This is consistent with previously published data, where this epitope was almost absent in nodule sections from Medicago. By contrast, (1-5)-α-L-arabinosyl, an epitope of RG-I that is recognized by LM6, is present in most cell walls of cells within the infection zone of the nodule (FIG. 6, Panel D) and predominantly accumulates around colonized cells of the fixation zone (FIG. 6, Panel D and FIG. 8), while uninfected cells within this zone did not accumulate (1-5)-α-L-arabinosyl (FIG. 8, Panels C′-C′″). To differentiate between HG subtypes, two antibodies were applied, LM20 and LM19, recognizing methyl-esterified and unesterified HGs, respectively. While esterified pectins were present in most cell walls (FIG. 1, Panel A), un-esterified pectins predominantly accumulated in epidermal cells, outer cortical cells, and around infection threads, while the cell wall of central uninfected and infected cells were devoid of this processed form of pectin (FIG. 1, Panel B). Furthermore, and as shown for nodular tissue, unesterified (FIG. 1, Panel C) but not methylesterified pectins (FIG. 1, Panel D) accumulated around ITs in root cortical cells. Labelling of unesterified pectins was also noted, frequently extended slightly from the ITs towards neighboring cells. These results provide insights into cell wall remodeling that takes place during rhizobial infections and transcellular IT passage and suggests the expression and activity of a pectin de-methylesterase during this process.

### Example 2: Dissecting Cell Wall Patterns at Transcellular IT Passage Sites

In order to further dissect cell wall patterns at transcellular IT passage sites, a correlative light-electron microscopy (CLEM) protocol was established. Briefly, CLEM was applied in 70 nm Lowicryl HM20 ultrathin section obtained with a Reichert-Jung ultramicrotome and collected in finder grids. The grids with the sections were washed with PBS buffer for 5 min, followed by an incubation with 0.12 M Glycine in PBS for 10 min. After 5 min washing in PBS, the grids were incubated for 10 min in blocking solution (4% BSA in PBS) followed by 30 min incubation with the first antibody in blocking solution. After six times washing for 3 min each in PBS, the grids were incubated for 30 min in blocking solution containing a fluorescence labelled second antibody. Grids were washed six times for 3 min each in PBS and incubated in 1% DAPI solution for 5 min before were mounted on a microscope glass slide for observation at the fluorescence microscope (ZEISS ApoTome.2). Additionally, immuno-gold staining was performed as described below for CLEM but substituting the second antibody by conjugated Protein A-gold and contrasting the sections after washing in water with 2% uranyl acetate.

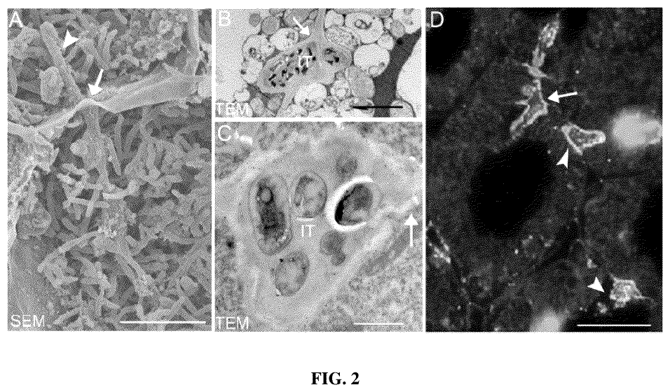

Using this correlative light-electron microscopy (CLEM) protocol allows searching for events by fluorescence microscopy (FIG. 1, Panels E, F) and later to perfectly retrieve these sites in ultrathin sections using transmission electron microscopy (TEM) (FIG. 1, Panels E′, F′). Overlaying those images revealed that unesterified pectins were present along the ITs and small segments of the host cell wall being in close proximity to the IT (FIG. 1, Panels E″, F″). These sites could be transcellular passage sites as frequently seen using scanning electron microscopy (SEM) (FIG. 2, Panel A) and TEM (FIG. 2, Panel B). Those observations were further confirmed by double immuno-gold labeling, which showed unesterified pectins (LM19, 12 nm) being concentrated at the IT penetration site, while only a few gold particles were detected using LM20 (methylesterified pectins, 5 nm) (FIG. 2, Panel C). These images also revealed that CW structures at transcellular IT passage sites fused and thickened (FIG. 2, Panel B). To assess whether this was a result of cell wall loosening and subsequent swelling or rather representing rigidified structures, these samples were probed using the 2F4 antibody, which recognizes ‘egg-box’ pectin dimers. 2F4 immunofluorescence indeed confirmed the accumulation of Ca²⁺-complexed pectin (FIG. 2, Panel D). This is in agreement with the observed enrichment of unesterified pectins around ITs, as Ca²⁺-complexation requires de-methylesterification of HGs (FIG. 1, Panels B, C).

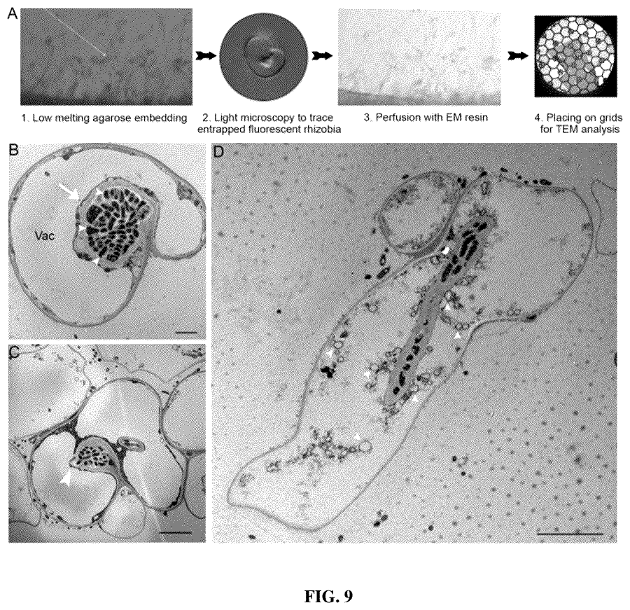

To visualize the cell wall of the IC and the IT in root hairs, a modified CLEM setup (FIG. 9, Panel A) was tested. In sharp contrast to nodular ITs, primary infections are rather rare and cannot be searched routinely by classical ultrathin sectioning and TEM. Thus, an infected root was embedded initially in low melting agarose and searched for curled root hairs using bacterial fluorescence before trimming the sample block and transferring into EM resin. Semi-thin sectioning was performed until the curled hair was reached. Ultra-thin serial sectioning was then subsequently performed for TEM analysis. As expected, the IC was surrounded by a thick and electron dense cell wall, that was much thinner at the possible IT initiation side (FIG. 9, Panels B-C). Sectioning of further samples also revealed such cell wall structures around root hair ITs that were additionally flanked by a multitude of vesicles (FIG. 9, Panel D). While this approach allowed an unprecedented view on IC and IT morphology in root hairs, it is limited in throughput. Thus, nodule samples were used in the following experiments.

### Example 3: Identification of a Symbiotic Pectin Methyl Esterase (SyPME)

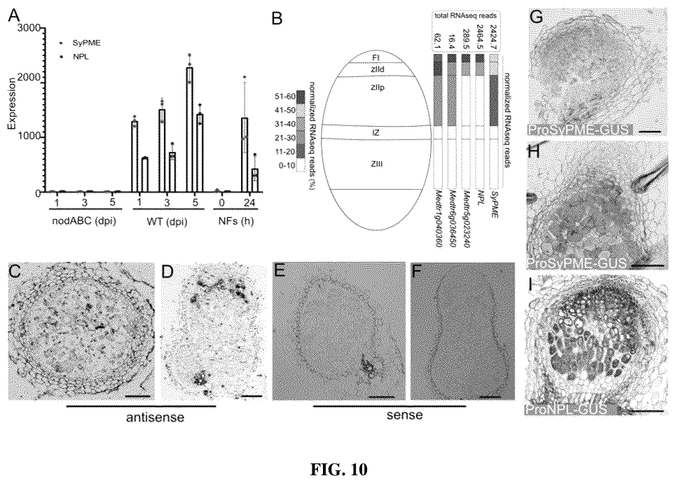

As described in Examples 1 and 2, unesterified pectins, but not methylesterified pectins, were present along the ITs and small segments of the host cell wall in close proximity to the IT. Pectin de-methylesterification is enzymatically mediated by pectin methyl esterases. The Medicago PME family, consisting of more than a hundred members, was searched for suitable candidates. Using published transcriptome data, one gene was identified (Medtr4g087980 or MtrunA17_Chr4g0069841) as being consistently induced upon Nod Factor application and S. meliloti inoculation in roots (FIG. 10, Panel A). The gene also remained highest expressed in the nodule meristem and in cells of the distal infection zone zIId (FIG. 10, Panel B). Thus, it was given the name ‘Symbiotic PME’ (SyPME). The transcriptome data was first verified by in situ hybridizations and expression of a transcriptional reporter using 2 kb upstream of the transcriptional start sites as a putative promoter region. When hybridizing nodule sections with an antisense in situ probe, SyPME transcripts were found in cortical cells of nodule primordia (FIG. 10, Panel C) and in zone zIId of mature nodules (FIG. 10, Panel D). No signals were observed when using the sense probes as control in the same tissues (FIG. 10, Panel E-F). Accordingly, SyPME promoter activity, as delineated by β-Glucuronidase (GUS)-staining, was found within the entire cortex of nodule primordia (FIG. 10, Panel F), while a confined expression domain limited to the infection zone II was observed in young and mature nodules (FIG. 10, Panel G). These results demonstrate the SyPME promoter's ability to modulate expression both spatially and temporally in developing and mature nodules and within the nodular infection zone of indeterminate nodules during symbiotic infection.

To assess the localization patterns of the SyPME protein, a SyPME-GFP translational fusion under the control of a Ubiquitin 10 promoter was generated and used for hairy root transformation.

Briefly, seeds of M. truncatula were washed 6 times with sterile tap water after being sterilized for 20 min with pure sulfuric acid (H₂SO₄). The seeds were then treated with bleaching solution (12% NaOCl, 0.1% SDS) for 60 s and washed again 6 times with sterile tap water. The sterilized seeds were covered with sterile tap water for 2 hours before being transferred to 1% agar plates and stratified at 4° C. for 3 days in darkness. After stratification, seeds were kept in dark at 24° C. for 24 hours for germination. The seed coat was removed, seeds were placed in a controlled environment chamber at 24° C. with a 16 h/8 h light/dark photoperiod and seedlings were then used for hairy root transformation as previously described (Boisson-Dernier et al., 2001). Composites were first placed onto solid Fahrdeus medium (containing 0.5 mM NH₄NO₃) and incubated in darkness (at 22° C.) for three days, following 4 days at 22° C. in white light with roots kept in the darkness. One week later, seedlings were transferred onto fresh Fahrdeus medium (0.5 mM NH₄NO₃) for another 10 days. Afterward, the transformed roots were screened and positive plants were transferred to open pots for phenotyping.

For phenotyping, seedlings were directly grown in open pots (mixture of 1:1 quarzsand:vermiculite mixture, 2 plants/pot) after seed germination. Plants were watered with liquid Fahrdeus medium (without nitrate, 30 ml/pot) and tap water (30 ml/pot) once a week, individually. The plants were then inoculated with S. meliloti (OD600=0.003) 7 days after transfer (20 ml/pot). Another 10 days later, plants were harvest for the quantification of infection structures.

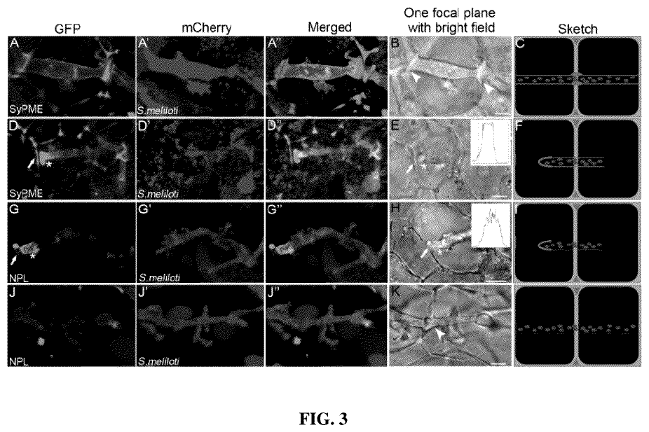

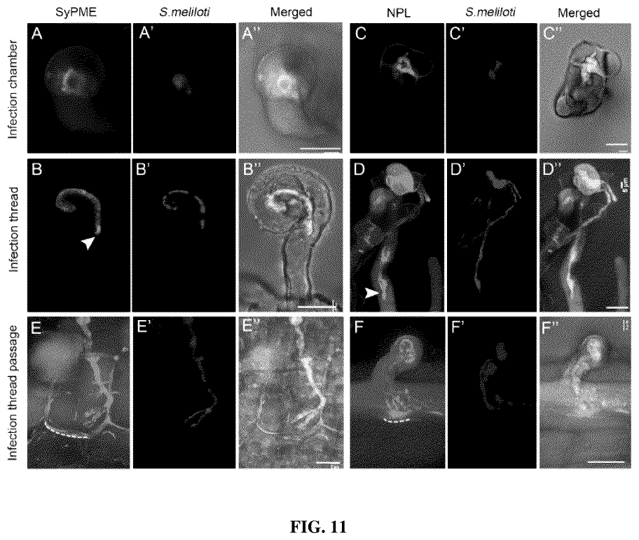

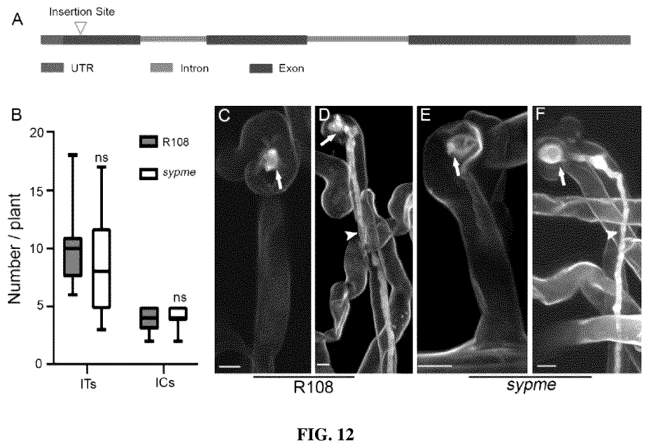

Clear and confined fluorescence was observed in root hairs around the infection chamber (FIG. 11, Panel A) and along growing primary ITs (FIG. 11, Panel B). In line with this, nodular ITs were also decorated by the SyPME protein (FIG. 3, Panel A), while the strongest accumulations were observed at transcellular passage sites (FIG. 3, Panels B, C). Here, SyPME localization was strictly delineated to the peripheral cell wall at cellular conjunctions with crossing ITs (FIG. 3, Panel B), thus sites that are rich in Ca²⁺-complexed unesterified pectins (FIG. 2, Panel D). Furthermore, SyPME also accumulated at both the tip region of growing ITs and a spatially confined site at the cell periphery that marks the site of the subsequent transcellular IT passage (FIG. 3, Panels D-F, FIG. 11, Panel E, and FIG. 12, Panels A-A′).

### Example 4: SyPME and NPL Cooperate to Regulate IT Growth

As unesterified pectins serve as substrates for pectin lyases (PLs) or polygalacturonases (PGs), the interplay between the Medicago Nodule Pectate Lyase (NPL; Medtr3g086320) and SyPME was assessed. SyPME and NPL are significantly co-expressed in infected root hairs (Correlation coefficient=0.9883, FIG. 10, Panel A). Compared to other members of the pectate lyase family being present in nodule transcriptomic data, NPL expression was found to be highest and mainly restricted to the nodule meristem and the zIId (FIG. 10, Panel B). This spatially controlled expression in nodules was also confirmed when generating a transcriptional GUS reporter using 2 kb (2038 bp) upstream of the NPL transcriptional start site (FIG. 10, Panel I). At protein level and in line with patterns observed for SyPME, a NPL-GFP fusion protein driven by the endogenous NPL promoter also localized to ICs, primary ITs in root hairs and the abovementioned spatially confined sites that will be penetrated by ITs (FIG. 11, Panels C-D, F). This indicates that SyPME precedes NPL-mediated pectin degradation at the IC, the tip region of growing ITs, and initially at the local cell wall site preparing for IT passage. Interestingly, older parts of these ITs showed reduced NPL accumulations (FIG. 11, Panel D) while SyPME protein levels remained high at these regions (FIG. 11, Panel B) suggesting a possible stiffening rather than loosening of the remnant ITs in root hairs. While NPL also localized to the tip of nodular ITs and local cell wall region near to nodular ITs, the protein was absent from transcellular passage sites itself (FIG. 3, Panels C, D). Consequently, unesterified pectins are, most likely, not degraded by NPL at these transcellular passage sites, which confirms that these regions are stabilized and possibly sealed by Ca²⁺-complexed pectins provided by SyPME function. This would equally restrict apoplastic spreading of rhizobia as well as interference with other microbes colonizing the intercellular space of plants grown in natural habitats. These experiments demonstrate the coordinated localization of SyPME and NPL during symbiotic infection.

### Example 5: A Genetic Framework Regulates IT Growth and Transcellular Passage

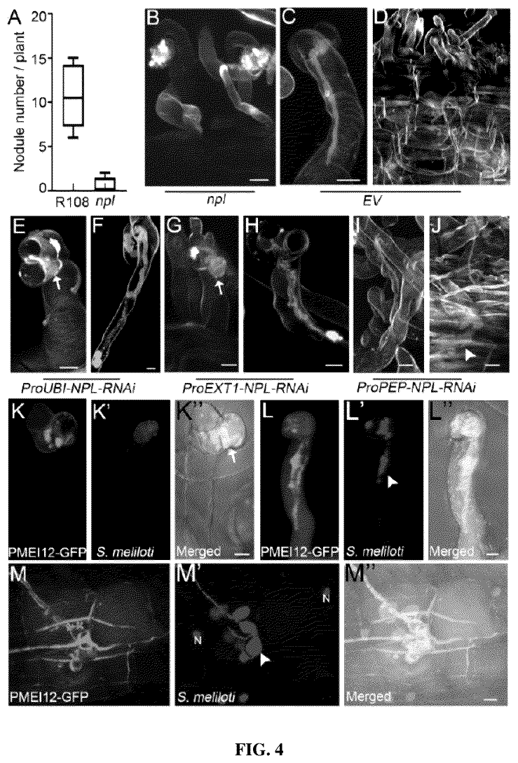

To analyze the cross-talk between SyPME and NPL in more detail, their impact on infection using loss- and gain-of-function approaches was assessed. Significantly less nodules formed on the Medicago npl mutant compared to R108 WT plants (FIG. 4, Panel A). Furthermore, most infection events were aborted at the IC stage (FIG. 4, Panel B), an observation which is consistent with previously published data. To be able to differentiate between the requirement of NPL in the epidermis and the root cortex, an RNA interference (RNAi) approach was also conducted expressing a silencing construct under the control of different promoters in transgenic roots. Briefly, roots transformed with empty vector and NPL-RNAi constructs were harvested 10 days post-inoculation, then fixed in PBS solution containing 4% PFA under vacuum for 15 min (twice) and kept at room temperature for 2 hours before being transferred to a Clearsee solution. Roots were kept in Clearsee for 2-3 days before the solution was refreshed and supplied with 0.1% Calcofluor white prior to imaging.

To generally prove the effectiveness of the construct used, an empty control vector was first expressed, where no changes in IT formation and progression were observed (EV; FIG. 4, Panel C, D). Constitutive over-expression of an NPL-RNAi construct, however, frequently resulted in trapped rhizobia within the IC (FIG. 4, Panel E) while only some ITs successfully elongated (FIG. 4, Panel F). The same pattern was observed when driving the silencing construct by the Solanum lycopersicum (tomato) expansin 1 (ProEXT1) promoter previously shown to mediate epidermis-specific expression in M. truncatula (FIG. 4, Panels G, H). By contrast, cortex-specific silencing of NPL upon using the Arabidopsis thaliana endopeptidase PEP-promoter predominantly resulted in normally developed ITs in infected root hairs that subsequently aborted in the root cortex (FIG. 4, Panels I, J). These results further demonstrate that NPL is required during both IT initiation and transcellular progression.

To genetically test whether SyPME and/or other members of this protein family are required for successful infections, the TNT1 transposon insertion collection was searched and a single sypme allele carrying an insertion in the first intron (NF2281 high 35; FIG. 12, Panel A) was identified. Homozygous individuals, however, did not show any symbiotic phenotypes (FIG. 12, Panels B-F), which might be due to the large size of this gene family. In order to target functionally redundant PMEs with spatio-temporal precision, the Arabidopsis thaliana PME INHIBITOR 12 (PMEI12), which has been previously demonstrated to efficiently inhibit PME activities, was expressed under control of the NPL promoter and inoculated these transgenic roots with S. meliloti. Indeed, the majority of infections were blocked at the IC stage (FIG. 4, Panels K-K″) or during IT progression in root hairs (FIG. 4, Panels L-L″) and the root cortex (FIG. 4, Panels M-M″), thus phenocopying the npl mutant. This supports the proposed dependency of NPL on preceding PME activity, and thus coordinated expression of SyPME throughout the infection process.

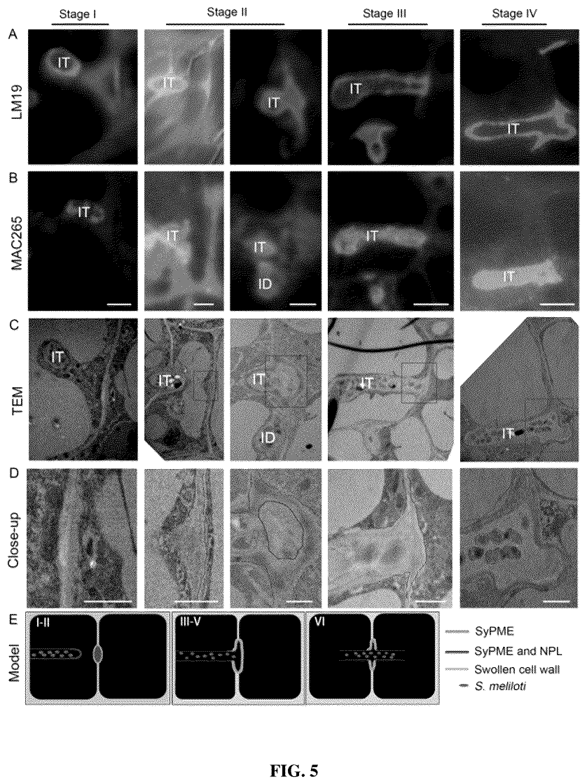

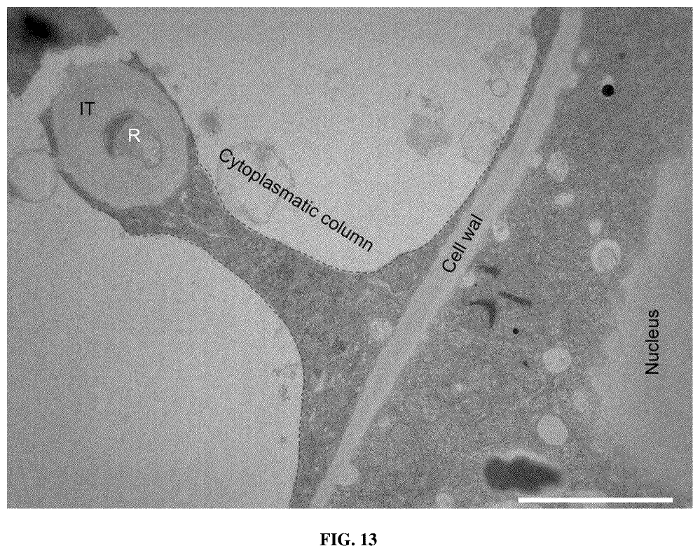

In an additional set of experiments, and to unambiguously unravel cell wall changes during transcellular IT passage, the CLEM approach was used to monitor pectins and alterations within the cell-cell interface at different stages of transcellular IT progression. For this, unesterified pectins (LM19, FIG. 5, Panel A) and the IT matrix (MAC265, FIG. 5, Panel B) were labeled and ultrathin sections (FIG. 5, Panels C-D) were searched for ITs approaching the basal cell membrane/cell wall and the moment of IT passage. Unesterified pectins accumulated around the future transcellular passage site, as defined by the cytoplasmatic column formed ahead of the IT, within the cell-cell interface (FIG. 5, stage I; FIG. 13). This passage site further increased in size, expanded laterally and subsequently swelled in the central region (FIG. 5, stage II). This was followed by rhizobia entering a closed apopolastic compartment prior to entry into the neighboring cell that already formed a pre-infection thread (FIG. 5, stage III). At the end, rhizobia entered the neighboring cell through the apopolastic space without any membrane confinement at this site (FIG. 5, stage IV).

Given the estimated diameter of the passage site in the range between 1 nm and 2 nm, the discontinuation of the IT membrane upon fusion of the IT with the basal membrane and the de novo invagination at the neighboring cell, transcellular passage is a successive series of events (FIG. 5E) that includes 1) targeted secretion of PMEs and initially of NPL, 2) local pectin de-methylesterification and partial pectin degradation at the tip of the IT and at the local host cell wall prepared for penetration, 3) maintenance of SyPME but reduction of NPL protein levels at the passage site, 4) confined cell wall swelling at the passage site, 5) spatial release of rhizobia into a sealed apoplastic compartment and 6) uptake into the neighboring cell.

* * * * * *

Having illustrated and described the principles of the present invention, it should be apparent to persons skilled in the art that the invention can be modified in arrangement and detail without departing from such principles. We claim all modifications that are within the spirit and scope of the claims. All publications and published patent documents cited herein are hereby incorporated by reference to the same extent as if each individual publication or patent application is specifically and individually indicated to be incorporated by reference.

What is claimed is:

1. A DNA molecule comprising a DNA sequence selected from the group consisting of:
a) a sequence having at least 85 percent sequence identity to SEQ ID NO: 1;
b) a sequence comprising SEQ ID NO: 1; and
c) a fragment of SEQ ID NO: 1 or a fragment having at least 85 percent sequence identity to the fragment of SEQ ID NO: 1, wherein the fragment has gene-regulatory activity;
wherein said sequence is operably linked to a heterologous transcribable polynucleotide molecule.

2. The DNA molecule of claim 1, wherein said DNA sequence has at least 90 percent sequence identity to the DNA sequence of any of SEQ ID NO: 1.

3. The DNA molecule of claim 1, wherein said DNA sequence has at least 95 percent sequence identity to the DNA sequence of any of SEQ ID NO: 1.

4. The DNA molecule of claim 1, wherein said fragment has at least 87 percent sequence identity to the fragment of SEQ ID NO: 1 if the fragment is less than 115 nucleotides.

5. The DNA molecule of claim 1, wherein the DNA sequence comprises gene regulatory activity.

6. The DNA molecule of claim 5, wherein the gene regulatory activity is promoter activity.

7. The DNA molecule of claim 5, wherein the gene regulatory activity is symbiotic-specific pectin methyl esterase (SyPME) promoter activity.

8. The DNA molecule of claim 1, wherein the heterologous transcribable polynucleotide molecule comprises a gene of agronomic interest.

9. The DNA molecule of claim 8, wherein the gene of agronomic interest is a gene encoding pectin methylesterase having pectin de-methylesterification activity.

10. The DNA molecule of claim 8, wherein the DNA sequence provides expression of said heterologous transcribable polynucleotide molecule in response to an external stimulus.

11. The DNA molecule of claim 10, wherein the DNA sequence provides expression of said heterologous transcribable polynucleotide molecule in a root hair cell, within the cortex of nodule primordia, a mature nodule, within a nodular infection zone in young, mature, or indeterminate nodules.

12. A transgenic plant cell comprising a heterologous DNA molecule comprising a sequence selected from the group consisting of:
a) a sequence having at least 85 percent sequence identity to any of SEQ ID NO: 1;
b) a sequence comprising any of SEQ ID NO: 1; and
c) a fragment of SEQ ID NO: 1 or a fragment having at least 85 percent sequence identity to the fragment of SEQ ID NO: 1, wherein the fragment has gene-regulatory activity;
wherein said sequence is operably linked to a heterologous transcribable polynucleotide molecule.

13. The transgenic plant cell of claim 12, wherein said transgenic plant cell is a monocotyledonous plant cell.

14. The transgenic plant cell of claim 12, wherein said transgenic plant cell is a dicotyledonous plant cell.

15. A transgenic plant, or part thereof, comprising the DNA molecule of claim 1.

16. A progeny plant of the transgenic plant of claim 15, or a part thereof, wherein the progeny plant or part thereof comprises said DNA molecule.

17. A transgenic seed, wherein the transgenic seed comprises the DNA molecule of claim 1.

18. A method of producing a commodity product comprising obtaining a transgenic plant or part thereof according to claim 15 and producing the commodity product therefrom.

19. The method of claim 18, wherein the commodity product is protein concentrate, protein isolate, grain, starch, seeds, meal, flour, biomass, or seed oil.

20. A commodity product comprising the DNA molecule of claim 1.

21. The commodity product of claim 20, wherein the commodity product is protein concentrate, protein isolate, grain, starch, seeds, meal, flour, biomass, or seed oil.

22. A method of expressing a transcribable polynucleotide molecule comprising obtaining a transgenic plant or part thereof according to claim 15 and cultivating the plant, wherein the transcribable polynucleotide molecule is expressed.


SEQUENCE LISTING (6 sequences)

SEQ ID NO: 1 - DNA, 2000 bp, Medicago truncatula
  source 1..2000: mol_type = genomic DNA
tgttctctctattaagattcctccctccccttcgttaaaccaaacaaaaagaagttgatatcactaagatgatagaagttgaatatttaatttagattagagtgcatgtcagcatcaaattattactaaaattaccgaaaccaaataattaaaaaacaaggcctgcatacaagaattggcataagagatggacgcggaaaaggaaagttaaaattgaaaagaaaagaaaacaacatattgaggaacctgccatacatggacaaacatgtgatgcttgtataaaaaagaaaagttctcctctcattttcattttcatttcatatatataaaatatggtgaaattgaatacgttactaaagtgaaaatttttaatttgatgaaatggactaagagagaaaaacaaaaatagatctagagccatctatgttgaggcagcaagctgtaataatcacattgggtgggtgacggtgtcgagaataatttacctcatgtattttgttctagatgattagtgaactgatctagagattttaattagtaaaatcagtatgtatatagaaatttatttttaataatagatggctctagatatgtttttgaaatgtactattagaaaaaacccaaatcccaaatcagatagataagacttttgagaagagtatataaagatgagacaatcctcaacttacaagctggttggttttgtaaggatgagttaaggggtttgaggcttttctttttttggaatataaattttaggctagttgttaacaaattttcttcttatataaatatatataaaaaatgtatatataatatcaagtaaaaattattgaaaatatactgctaaaaaaataattattgaaaatttatagtgggttacttgtcatcacatcttttcaaataagtcgattaatgtattatggtagaagttgtaaagagaaagttcatatagagagaaaaagaatggatgttttcttttgaagaacctaggaacgaaagtcaaaagaacaaaatgcttaaaagtgaaaaattacaggtattagagatactctaaaaaaattagaatatgtatcatcgtgaacaaactcataaacattccgaaggctccttcaaaaaaacattctgaaggtaatcgatataaaaacacgatgacataatgatttattttagtaaacgatccattcatttgcgtgatagatttattaaaagaattgtgaatttacctcatgtattttgatagagaaatttaaccttgagattagcttagttttaaatagtgtaattgccgccgaacttgacaacgtaaacttgtgatctaagtagaagccttaagatgttggaatgtcctttattatgtgtcaaaaaagaaatagtgtgattgaaaaaaaaaatcattgatttaattatatcttaagatgtggtgtgttatgaaaatataacgaaagaatgacatgacaatacaatatttaatatccctaaaaaaaaatacaatatttagtagaagagttcttgattttatagagttaaataattttatattaattaaaactaagattgaaatatacacatatactatcactaaaaaaagaaaagaaatatatacatatacatttatatgagcataaattgttttggagactaaacttttcttgttaaaaattgaaatgtgtttgtaaagtttaattcaataaataatgtcgagattgttatactaaatatttcatgtgatattaaagtttagatttataatatctgatctatatgtcttcatttttattgacgtttatagtttcacctacgtttaaaattactataaaagttagaatacaaagtttattagcatctaaccagctctacacaactgaatctacaatcttttccttcctttaaataatctcaacatctctcaattcttatttgcctcttaacaaaataaaaaccatagaattagtgacacttacttactcatcgtgtctattatatttcaaagttctaaa

SEQ ID NO: 2 - DNA, 1557 bp, Medicago truncatula
  source 1..1557: mol_type = genomic DNA
atggaactatgttcctcaaaacagccatccacaacaacactctcaataactacattcatcttcttcttcctcctcctcctctcactcccacactccacttttgcttcttctcccaatgactttgttggttcatccctcagagtttctaccgccaaatttgccaactctgctgaagaagttatcaccgttttacaaaaagtcatctctattttatcacgcttcactaatgtctttggtcactctcgcacctcaaacgccgtttcggattgcctcgatttacttgacatgtccttagaccaacttaaccagtccatttccgctgctcaaaagcccaaagagaaggataacagcacaggaaaactaaactgtgatctaaggacatggctgagtgctgttcttgtgtatccagacacatgtatagaaggattagaaggttcaattgtaaagggtcttatatcctctggtctcgatcatgtaatgtccttggtggcgaatcttcttggtgaagtagtttccggcaatgatgatcaactcgcaaccaacaaggatcgatttccttcgtggatcagagatgaggacacaaagttgttgcaggcaaatggtgtgacggctgatgctgttgtggctgctgatggaagtggagactacgctaaggtgatggatgccgtatcggcagcacctgaaagcagcatgaaaagatatgtaatatatgtgaaaaaaggtgtttatgttgaaaatgttgagattaagaagaagaaatggaatatcatgttgattggtgaaggcatggatgctactatcatctccggcagtcggaattatgtggacggttcgacaactttccggtcagctacctttgctgtaagtggtagagggttcatagcacgcgacatttcatttcagaacaccgcaggtgccgagaagcaccaagcagtggcgttaagatcagactctgacctctcagtattttatcggtgtgggatattcggttatcaagatagcctatatactcacaccatgcgtcagttctaccgtgaatgcaaaattagtggtactgtggatttcatctttggagacgccacggctgtgttccaaaactgtcaaatactagccaagaaagggatgcctaaacaaaagaacactgttactgctcaaggacgaaaagacccgaaccaaccaactggtttctccttccaattttgcaacatttccgcagattctgaccttctaccatcggtcacaaccatcccaacataccttggtagaccctggaagacttattcgcgaacaatttttatgcaatcctacatgagtgacgcaataaggccagaagggtggttagagtggaacggtaattttgctttgaacacattgtactacgctgaatacatgaactctggaccgggtgccggggtagctaaccgggtgaaatggtcaggataccatgtattgaatgattcgagtgaggctactaaattcactgtggctcaatttattgaggggaatctttggttaccttccaccggtgtaacttacacttctggtttgaaagtgtaa

SEQ ID NO: 3 - protein, 518 aa, Medicago truncatula
MELCSSKQPSTTTLSITTFIFFFLLLLSLPHSTFASSPNDFVGSSLRVSTAKFANSAEEVITVLQKVISILSRFTNVFGHSRTSNAVSDCLDLLDMSLDQLNQSISAAQKPKEKDNSTGKLNCDLRTWLSAVLVYPDTCIEGLEGSIVKGLISSGLDHVMSLVANLLGEVVSGNDDQLATNKDRFPSWIRDEDTKLLQANGVTADAVVAADGSGDYAKVMDAVSAAPESSMKRYVIYVKKGVYVENVEIKKKKWNIMLIGEGMDATIISGSRNYVDGSTTFRSATFAVSGRGFIARDISFQNTAGAEKHQAVALRSDSDLSVFYRCGIFGYQDSLYTHTMRQFYRECKISGTVDFIFGDATAVFQNCQILAKKGMPKQKNTVTAQGRKDPNQPTGFSFQFCNISADSDLLPSVTTIPTYLGRPWKTYSRTIFMQSYMSDAIRPEGWLEWNGNFALNTLYYAEYMNSGPGAGVANRVKWSGYHVLNDSSEATKFTVAQFIEGNLWLPSTGVTYTSGLKV

SEQ ID NO: 4 - DNA, 618 bp, Medicago truncatula
  source 1..618: mol_type = genomic DNA
atggaagaatcgaaaaacaaacaattagagttagttgacaccctaactccactacctcaatctgaatctgaacctcgagagttttcctacttccttgaagaaaaggaaccggggaacgaaggaacttcaagcagtgttgttaaacaagagcgtgtcgtgagtgatcatgctacttcttcagttgatcaaactactgctgctggaacagatacaaaggactcagttgaccgagatgctgtactagcaagggttgaatcgcagaaaagattggctttaattaaagcatgggaagaaaatgagaagacaaaagtagagaatagggcatataagatgcaatctgctgttgatttgtgggaggatgataagaaagcatcaattgaagcaaaattcaaaggaattgaggtaaaattggacagaaagaagagtgaatatgttgaagtaatgcaaaacaaaataggtgaaattcataaatcagcagaagagaaaaaggcaatgatagaagctcaaaaaggggaagaaatcctcaaggtagaggagacagctgcaaagtttcgtactcgtggctatcagccaagaagacttcttggatgtttcagcggtttaaggtttttcagttag

SEQ ID NO: 5 - protein, 615 aa, Medicago truncatula
METGLUGLUSERLYSASNLYSGLNLEUGLULEUVALASPTHRLEUTHRPROLEUPROGLNSERGLUSERGLUPROARGGLUPHESERTYRPHELEUGLUGLULYSGLUPROGLYASNGLUGLYTHRSERSERSERVALVALLYSGLNGLUARGVALVALSERASPHISALATHRSERSERVALASPGLNTHRTHRALAALAGLYTHRASPTHRLYSASPSERVALASPARGASPALAVALLEUALAARGVALGLUSERGLNLYSARGLEUALALEUILELYSALATRPGLUGLUASNGLULYSTHRLYSVALGLUASNARGALATYRLYSMETGLNSERALAVALASPLEUTRPGLUASPASPLYSLYSALASERILEGLUALALYSPHELYSGLYILEGLUVALLYSLEUASPARGLYSLYSSERGLUTYRVALGLUVALMETGLNASNLYSILEGLYGLUILEHISLYSSERALAGLUGLULYSLYSALAMETILEGLUALAGLNLYSGLYGLUGLUILELEULYSVALGLUGLUTHRALAALALYSPHEARGTHRARGGLYTYRGLNPROARGARGLEULEUGLYCYSPHESERGLYLEUARGPHEPHESER

SEQ ID NO: 6 - DNA, 1557 bp, Medicago truncatula; identical to SEQ ID NO: 2
  source 1..1557: mol_type = genomic DNA